(19) United States
(12) Patent Application Publication
(10) Pub. No.: US 2019/0209815 A1
(43) Pub. Date: Jul. 11, 2019

(54) Catheter for Applying a Medium Into the Middle Ear

(71) Applicants: David Spiggle, Neunkirchen-Seelscheid (DE); Detlef Theis, Wiehl (DE)

(72) Inventors: David Spiggle, Neunkirchen-Seelscheid (DE); Detlef Theis, Wiehl (DE)

(21) Appl. No.: 16/331,551

(22) PCT Filed: Sep. 14, 2017
(86) PCT No.: PCT/DE2017/100777

§ 371 Date: Mar. 8, 2019

(30) Foreign Application Priority Data
Sep. 16, 2016 (DE) 10 2016 117 460.2

(51) Int. Cl. A61M 25/10; A61F 11/00; A61M 25/00

(52) U.S. Cl. CPC A61M 25/1011; A61M 2025/0042; A61M 25/0026; A61F 11/002

(57) ABSTRACT

A catheter for applying a medium into the middle ear, includes a shaft having a distal and a proximal end, an inflatable first balloon, the interior of the first balloon being in communication with a first inflation lumen, an inflatable second balloon which is arranged distally in relation to the first balloon, the interior of the second balloon being in communication with a second inflation lumen, an application lumen having an inlet opening, an outlet opening and a center section that extends from the inlet opening to the outlet opening, the inlet opening being arranged outside of the shaft, the center section extending at least partially through the shaft and the outlet opening being arranged distally in relation to the proximal end of the second balloon, as well as a vent lumen having an inlet opening, an outlet opening and a center section that extends from the inlet opening to the outlet opening, the inlet opening being arranged distally in relation to the proximal end of the second balloon, the center section extending at least partially through the shaft and the outlet opening being arranged outside of the shaft.

The invention relates to a catheter for applying a medium into the middle ear.

The middle ear is connected to the nose and throat via the Eustachian tube, with the Eustachian tube allowing pressure equalization between these areas. Since the middle ear is difficult to access through the narrow Eustachian tube, application into the middle ear are usually made by injections through the eardrum.

Application catheters are known in the prior art. These application catheters known in the prior art are predominantly used in urology, radiology and other medical fields, but are not suitable for applying a medium into the middle ear.

A disadvantage of the application catheters known from the prior art is that they can not be placed accurately, that they may damage the auditory ossicles in the middle ear, that they do not seal the Eustachian tube, and that they only allow for applying a small volume of medium, if any.

The object of the invention is to provide a catheter which allows for an exact placement of the distal end of the catheter within the tympanic cavity, which minimizes the risk of ossicular damage, which allows for applying a medium and which prevents the medium from being discharged via the Eustachian tube.

To achieve the object, there is provided a catheter for applying a medium into the middle ear comprising a shaft having a distal and a proximal end, an inflatable first balloon, the interior of the first balloon being in communication with a first inflation lumen, an inflatable second balloon which is arranged distally in relation to the first balloon, the interior of the second balloon being in communication with a second inflation lumen, an application lumen having an inlet opening, an outlet opening and a center section that extends from the inlet opening to the outlet opening, the inlet opening being arranged outside of the shaft, the center section extending at least partially through the shaft and the outlet opening being arranged distally in relation to the proximal end of the second balloon, as well as a vent lumen having an inlet opening, an outlet opening and a center section that extends from the inlet opening to the outlet opening, the inlet opening being arranged distally in relation to the proximal end of the second balloon, the center section extending at least partially through the shaft and the outlet opening being arranged outside of the shaft.

The advantages achieved by the invention include, inter alia, the fact that the first, proximal balloon seals the Eustachian tube after inflation, so that the applied medium remains in the middle ear. In addition, the catheter is stabilized by this balloon in its position. The inflated second distal balloon prevents the distal end of the catheter from being pulled out of the middle ear by inflation of the proximal balloon back into the Eustachian tube and also protects the auditory ossicles from damage, for example, by the distal end of the shaft. In addition, the distal balloon also seals off the Eustachian tube. A medium can be applied by means of the application lumen. Surprisingly, it has also been found that the application of media is greatly facilitated when a vent lumen is present, which ensures the venting of the middle ear during the application. Further advantages of the invention will become apparent from the dependent claims.

The catheter is provided for applying media, in particular liquids or suspensions into the middle ear, in particular to the membranes leading to the inner ear. For this purpose, the catheter is pushed, with the distal end of the shaft first, by means of an insertion tool from the opening of the auditory tube through the Eustachian tube until the distal end of the shaft including the second, distal balloon has reached the tympanic cavity. In this position the first, proximal balloon is at least partially within the Eustachian tube. In this position, the catheter is fixed by inflating the first balloon and the second balloon and the application medium is injected through the application lumen while allowing air to escape through the vent lumen. The insertion tool is removable via the proximal end of the shaft. During the application, it is advantageous if the head of the patient is positioned laterally so that the ear to be treated points in the direction of the floor with the auricle. The application and ventilation is thus based on the “shower head principle”.

Thus, according to the invention, the distal end of the shaft means the end which is first pushed into the Eustachian tube. As a result, the balloon having the shortest distance to the distal end of the shaft is called the distal balloon. The other end of the shaft opposite the distal end thus forms the proximal end of the shaft. According to the invention, an article is referred to as proximal when it is arranged from the respective reference point in the direction of the proximal end of the shaft. An article is referred to as distal according to the invention when it is arranged from the respective reference point in the direction of the distal end of the shaft.

According to the invention, the catheter thus comprises a shaft having a distal and a proximal end. The shaft envelops at least a section of the lumens of the catheter.

The catheter further comprises an inflatable first balloon. The interior of the first balloon is in communication with a first inflation lumen. In addition, the catheter comprises an inflatable second balloon, the interior of which is in communication with a second inflation lumen. The second balloon is arranged distally in relation to the first balloon.

The first and second balloons of the catheter according to the invention may be inflated, I e., expanded, by introducing fluids, preferably liquids, more preferably saline, in particular isotonic NaCl solution (0.9%), through the lumens connected to the balloons, referred to according to the invention as inflation lumens. For deflation, the inflation media can be discharged through these lumens connected to the balloons.

The first and second inflation lumens extend from the first and second balloons, respectively, to their proximal openings being arranged outside of the shaft. The first and second inflation lumens may merge, inside or outside the shaft, into a common inflation lumen so that they share a common proximal opening. Preferably, the first and second inflation lumens or the common inflation lumen extend from the first and second balloons within the shaft toward the proximal end of the shaft and run out of the shaft through at least one shaft opening, preferably at least one shaft opening at the proximal end of the shaft.

The application lumen has an inlet opening, an outlet opening and a center section that extends from the inlet opening to the outlet opening. The inlet opening is arranged outside of the shaft. Starting from the inlet opening, the middle section extends through a shaft opening, preferably at the proximal end of the shaft, into the shaft interior, the center section extends inside the shaft in the direction of the distal end of the shaft and exits through a shaft opening out of the shaft interior and ends in the outlet opening, which is arranged distally in relation to the proximal end of the second balloon. The media to be applied can be introduced into the inlet opening, run through the middle section and exit the lumen at the outlet opening.

According to the invention, suitable media are preferably liquids and suspensions. Particularly preferred are biologically active compounds, very particularly preferably medicaments.

The vent lumen has an inlet opening, an outlet opening and a center section which extends from the inlet opening to the outlet opening. The outlet opening is arranged outside of the shaft. Starting from the outlet opening the center section extends through a shaft opening, preferably at the proximal end of the shaft, into the shaft interior, the center section extends further within the shaft toward the distal end of the shaft and exits through a shaft opening from the shaft interior and ends in the inlet opening, which is arranged distally in relation to the proximal end of the second balloon.

The vent lumen serves to vent the middle ear during the application and ensures that there is no overpressure in the inner ear, which prevents the application of the application medium or damages components of the middle ear. Air, application medium or other liquids located in the middle ear can thus enter the inlet opening through this vent lumen, pass through the center section to the outlet opening and exit through the outlet opening. The outlet opening of the application lumen and the inlet opening of the vent lumen are located in an area of the shaft that extends from the proximal end of the second, i. e., distal balloon to the distal end of the shaft. Thus, the application lumen and the vent lumen terminate at the level of the second balloon or distally in relation to said second balloon.

The first inflation lumen, the second inflation lumen, or the common inflation lumen in the area external to the shaft, as well as the application lumen and vent lumen in the areas extending outside of the shaft toward the inlet opening in the case of the application lumen and in the direction of the outlet opening in the case of the vent lumen, may have a device, such as a valve, a slide, a flap or a tap, by means of which the flow rate is adjustable. All lumens may also have connectivity for catheter couplings in these areas outside the shaft.

In addition to the first inflation lumens, second inflation lumens, application lumens, and vent lumens described above, the catheter may also comprise additional lumens. It is also possible that the catheter according to the invention comprises further balloons in addition to the first and second balloons.

In an advantageous configuration of the invention, the first inflation lumen and the second inflation lumen are not in fluid communication with each other. Accordingly, the first and second balloons are each inflatable by means of a separate lumen. This allows the second balloon to be inflated first and then the first balloon to be inflated, whereby the distal end of the shaft can not slip back into the Eustachian tube once the proximal first balloon is inflated.

In an advantageous configuration of the invention, the catheter thus has a total of four lumens. The first and the second balloons, respectively, can be inflated separately by means of the first and second inflation lumens, respectively, media can be applied by means of the application lumen, and the middle ear can be vented by means of the vent lumen during the application.

In an advantageous configuration of the invention, the outlet opening of the application lumen and the inlet opening of the vent lumen are arranged at the distal end of the shaft. In such a configuration, the center section of these lumens terminates in shaft openings at the distal end of the shaft. This arrangement ensures that, in the event that the patient's ear to be treated does not point in the direction of the floor with the auricle during or after application of the medium and the medium thus collects in the tympanic cavity around the second, distal balloon, this collected medium can not escape directly through the vent lumen or the application lumen.

In an advantageous configuration of the invention, the application lumen and the vent lumen extend from the shaft openings at the distal end of the shaft to the proximal end of the shaft within the shaft, and terminate at the inlet opening in the case of the application lumen, or with the outlet opening in the case of the vent lumen, outside the proximal end of the shaft, wherein the outlet opening in the case of the vent lumen, or the inlet opening in the case of the application lumen are arranged at the shaft openings at the distal end of the shaft.

In an advantageous configuration of the invention, the distal balloon, which according to the invention is referred to as the second balloon, is arranged at the distal end of the shaft. In such an advantageous configuration, the outlet opening of the application lumen and the inlet opening of the vent lumen as well as the second balloon are thus located at the distal end of the shaft. According to the invention, a balloon is arranged at the distal end of the shaft when, in the inflated state, it extends at least at one end along the longitudinal axis of the shaft to the distal end of the shaft. Such a configuration has the advantage that, due to the distally arranged balloon, no catheter components protrude, which could damage the middle ear.

In an advantageous configuration of the invention, the first and second balloons completely envelop the lateral surface of the shaft in the area in which they extend along the longitudinal axis of the shaft.

Surprisingly, it has been found that the application of a medium into the middle ear is considerably facilitated if the vent lumen has a larger diameter than the application lumen. In an advantageous configuration of the invention, the vent lumen therefore has a larger diameter than the application lumen. Preferably, the diameter of the vent lumen is at least 5%, particularly preferably at least 10%, and very particularly preferably at least 20% larger than the diameter of the application lumen. In an advantageous embodiment of the invention, the catheter lumen each have a diameter between 0.05-0.5 mm.

In an advantageous configuration of the invention, the shaft diameter is less than 2 mm, preferably less than 1.5 mm, particularly preferably less than 1.2 mm, in particular less than 1 mm. According to the invention, shaft diameter is understood to be the diameter perpendicular to the longitudinal axis of the shaft. A catheter with such a small shaft diameter ensures that the catheter can be inserted without great pressure and pushed through the Eustachian tube.

In an advantageous configuration of the invention, the first and/or second balloons are low-pressure balloons. According to the invention, the catheter thus comprises first and/or second balloons which are inflatable with less than 1 bar, preferably less than 0.75 bar, very particularly preferably less than 0.5 bar gauge. Such low-pressure balloons have the advantage over high-pressure balloons that they better protect the mucosa of the Eustachian tube from damage, better seal the Eustachian tube due to the increased flexibility of the balloons and ensure adequate stability.

In an advantageous configuration of the invention, the first balloon in the inflated state has a length of 5 to 30 mm, preferably 8 to 20 mm, particularly preferably 10 to 16 mm, and/or a diameter of 3 to 12 mm, preferably 5-10 mm, particularly preferably 7-8 mm. In an advantageous configuration of the invention, the second balloon in the inflated state has a length of 2 to 8 mm, preferably 4 to 6 mm, and/or a diameter of 2 to 8 mm, preferably 4 to 6 mm. According to the invention, the length of the balloons is measured parallel to the longitudinal axis of the catheter, while the diameter is measured perpendicular to the longitudinal axis of the catheter.

In an advantageous configuration of the invention, the first balloon is spaced 3-10 mm, preferably 5-8 mm, from the second balloon. In order to determine the distance, according to the invention, the distance is measured between the distal end of the first balloon and the proximal end of the second balloon in the inflated state. In such a configuration of the invention, the balloons are thus separated from each other by the shaft, comprising the application lumen, the vent lumen, and the inflation lumen for the second balloon. In this way, the catheter can both be fixed well in the application position and seal the Eustachian tube.

In a further configuration of the invention, the first balloon can be connected to the second balloon by means of a web, so that the first and second balloons are formed by the same balloon envelope. A web is thus understood to mean the balloon envelope, which envelopes the shaft in the area between the distal end of the first balloon and the proximal end of the second balloon. In such a configuration the diameter of the shaft including the web is, in balloons in the inflated state by not more than 15%, preferably not more than 10%, particularly preferably not more than 5%, in particular 0.5% greater than the shaft diameter excluding the web in this area.

According to the invention, the balloons can have various shapes. In an advantageous configuration of the invention, the second, distal balloon in the inflated state has a shape in which the diameter of the proximal end of the balloon is smaller than the diameter of the distal end of the balloon. Preferably, the second balloon in the shape of a cone, a truncated cone, a pyramid or a truncated pyramid, wherein the tip or the top surface faces towards the proximal end of the shaft. Such or similar shapes offer the advantage that the distal end of the catheter automatically draws into the tympanic cavity upon inflation of the balloon.

In an advantageous configuration of the invention, the second balloon in the inflated state extends along the longitudinal axis of the shaft partially beyond the distal end of the shaft. In such a catheter, the outlet opening of the application lumen and the inlet opening of the vent lumen, when viewing the shaft perpendicularly to the longitudinal axis, are recessed behind the balloon section which extends beyond the distal end of the catheter. This provides a further improved protective effect, so that the risk of injury to the auditory ossicles is minimized.

The shaft according to the invention can also have x-ray markers by means of which the position of the shaft can be determined. Preferably, these markers are located at the distal end of the shaft, between the first and second balloons, and/or proximally in relation to the second balloon.

Thus, the invention is directed to a catheter for applying a medium into the middle ear comprising a shaft having a distal and a proximal end, an inflatable first balloon, the interior of the first balloon being in communication with a first inflation lumen, an inflatable second balloon which is arranged distally in relation to the first balloon, the interior of the second balloon being in communication with a second inflation lumen, an application lumen having an inlet opening, an outlet opening and a center section that extends from the inlet opening to the outlet opening, the inlet opening being arranged outside of the shaft, the center section extending at least partially through the shaft and the outlet opening being arranged distally in relation to the proximal end of the second balloon, as well as a vent lumen having an inlet opening, an outlet opening and a center section that extends from the inlet opening to the outlet opening, the inlet opening being arranged distally in relation to the proximal end of the second balloon, the center section extending at least partially through the shaft and the outlet opening being arranged outside of the shaft
wherein the first inflation lumen and the second inflation lumen are not in fluid communication with each other,
wherein the first and second balloons are inflatable by means of one lumen each,
wherein the outlet opening of the application lumen and the inlet opening of the vent lumen are arranged at the distal end of the shaft,
wherein the second balloon is arranged at the distal end of the shaft,
wherein the vent lumen has a larger diameter than the application lumen
wherein the shaft diameter is less than 2 mm, particularly preferably less than 1.5 mm, particularly preferably less than 1.2 mm, in particular less than 1 mm,
wherein the first and/or second balloons are inflatable with less than 1 bar, preferably less than 0.75 bar, very particularly preferably less than 0.5 bar gauge,
wherein the first balloon in the inflated state has a length of 5 to 30 mm, preferably 8 to 20 mm, particularly preferably 10 to 16 mm, and/or a diameter of 3 to 12 mm, preferably of 5-10 mm, particularly preferably 7-8 mm,
wherein the second balloon in the inflated state has a length of 2 to 8 mm, preferably of 4 to 6 mm, and/or a diameter of 2 to 8 mm, preferably 4 to 6 mm,
wherein the first balloon is spaced 3-10 mm, preferably 5-8 mm from the second balloon,
wherein the first and second balloons are interconnected by means of a web,
wherein the second balloon in the inflated state has a shape in which the diameter of the proximal end of the balloon is less than the diameter of the distal end of the balloon, and/or
wherein the second balloon in the inflated state extends partially beyond the distal end of the shaft along the longitudinal axis of the shaft.

To solve the problem, a catheter for applying a medium is thus provided, comprising a first inflation lumen, a second inflation lumen, an application lumen and a vent lumen, a first balloon, a second balloon, which is arranged distally in relation to the first balloon, wherein the first balloon is inflatable by means of the first inflation lumen and the second balloon is inflatable by means of the second inflation lumen, and wherein the application lumen and the vent lumen extend to the distal end of the catheter.

The invention will be explained again below with reference to figures.

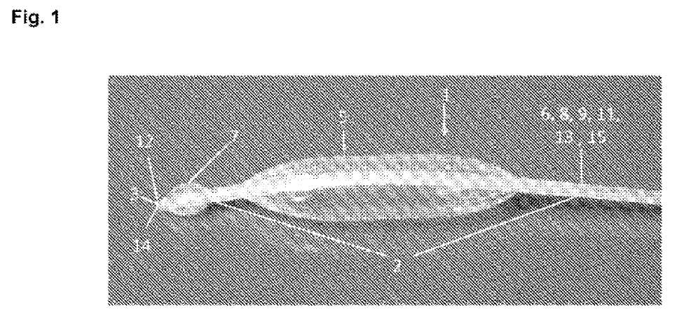

FIG. 1 shows a partial section of an embodiment of the catheter according to the invention.

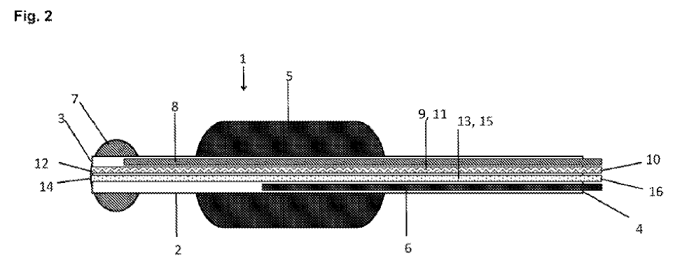

FIG. 2 shows a schematic representation of a cross section along the longitudinal axis of an embodiment of the catheter according to the invention.

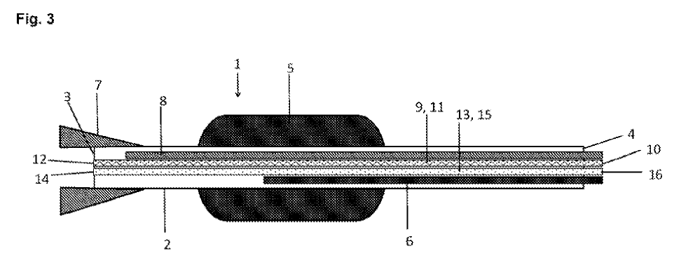

FIG. 3 shows a schematic representation of a cross section along the longitudinal axis of an embodiment of the catheter according to the invention.

FIG. 1 shows a section of an embodiment of the catheter 1 according to the invention, which comprises a shaft 2 having a distal end of the shaft 3 and a proximal end of the shaft 4, not shown. In the illustrated embodiment, the catheter 1 comprises an inflatable first balloon 5, the interior of which is in communication with a first inflation lumen 6. Distally in relation to the first balloon 5, an inflatable second balloon 7 is arranged, the interior of which is in communication with a second inflation lumen 8. The first and second inflation lumens extend within the illustrated catheter section within the shaft 2. In addition, the catheter has an application lumen 9 with an inlet opening 10, not shown, outside the shaft, an outlet opening 12 and a center section 11 which extends from the inlet opening to the outlet opening. The outlet opening 12 of the application lumen is arranged distally in relation to the proximal end of the second balloon 7. Likewise, the catheter 1 comprises a vent lumen 13 with an inlet opening 14 which is arranged distally in relation to the proximal end of the second balloon 7. The middle section 15 extends between the inlet opening 14 and the outlet opening 16, not shown, outside the shaft.

FIG. 2 shows a schematic cross-sectional view of a catheter 1 according to the invention along the longitudinal axis of the catheter 1, which comprises a shaft 2 having a distal end 3 and a proximal end 4. The catheter 1 further comprises a first balloon 5, which is inflatable by means of a first inflation lumen 6, and a second balloon 7, which is inflatable by means of a second inflation lumen 8. The outlet opening of the application lumen 9 and the inlet opening of the vent lumen 14 are arranged distally in relation to the proximal end of the second balloon 7. From the outlet opening of the application lumen 12, the center section of the application lumen 11 extends through the proximal end of the shaft and ends in the inlet opening 10. The center section of the vent lumen 15 extends from the inlet opening of the vent lumen 14 through the proximal end of the shaft and terminates in the outlet opening 16.

FIG. 3 shows a schematic cross-sectional view of a catheter 1 according to the invention as shown in FIG. 2, in which the second balloon 7 in the inflated state has a shape in which the diameter of the proximal end of the balloon is less than the diameter of the distal end of the balloon. The second balloon 7 extends in the inflated state, as shown, in the longitudinal direction of the catheter partially beyond the distal end of the shaft 3.

### LIST OF REFERENCE NUMERALS

1 Catheter
2 Shaft
3 Distal end of the shaft
4 Proximal end of the shaft
5 First balloon
6 First inflation lumen
7 Second balloon
8 Second inflation lumen
9 Application lumen
10 Inlet opening of the application lumen
11 Center section of the application lumen
12 Outlet opening of the application lumen
13 Vent lumen
14 Inlet opening of the vent lumen
15 Center section of the vent lumen
16 Outlet opening of the vent lumen

1. A catheter for applying a medium into the middle ear, comprising:
a shaft having a distal and a proximal end,
an inflatable first balloon, the interior of the first balloon being in communication with a first inflation lumen,
an inflatable second balloon which is arranged distally in relation to the first balloon, the interior of the second balloon being in communication with a second inflation lumen,
an application lumen having an inlet opening, an outlet opening and a center section that extends from the inlet opening to the outlet opening, the inlet opening being arranged outside of the shaft, the center section extending at least partially through the shaft and the outlet opening being arranged distally in relation to the proximal end of the second balloon, and
a vent lumen having an inlet opening, an outlet opening and a center section that extends from the inlet opening to the outlet opening, the inlet opening being arranged distally in relation to the proximal end of the second balloon, the center section extending at least partially through the shaft and the outlet opening being arranged outside of the shaft,
wherein the outlet opening of the application lumen and the inlet opening of the vent lumen are arranged at the distal end of the shaft.

2. The catheter of claim 1, wherein the first inflation lumen and the second inflation lumen are not in fluid communication with each other.

3. The catheter of claim 1, wherein the second balloon is arranged at the distal end of the shaft.

4. A catheter according to claim 1, wherein the vent lumen has a larger diameter than the application lumen.

5. The catheter according to claim 1, wherein the shaft diameter is less than 2 mm, preferably less than 1.5 mm, particularly preferably less than 1.2 mm, in particular less than 1 mm.

6. The catheter according to claim 1, wherein the first and/or second balloons are inflatable with less than 1 bar, preferably less than 0.75 bar, very particularly preferably less than 0.5 bar gauge.

7. The catheter according to claim 1, wherein the first balloon in the inflated state has a length of 5 to 30 mm, preferably 8 to 20 mm, particularly preferably 10 to 16 mm.

8. The catheter according to claim 1, wherein the second balloon in the inflated state has a length of 2 to 8 mm, preferably 4 to 6 mm, and/or the second balloon in the inflated state has a diameter of 2 to 8 mm, preferably 4 to 6 mm.

9. The catheter according to claim 1, wherein the first balloon is spaced 3-10 mm, preferably 5-8 mm from the second balloon.

10. The catheter according to claim 1, wherein the first and second balloons are interconnected by moans of a web.

11. The catheter according to claim 1, wherein the second balloon in the inflated state has a shape in which the diameter of the proximal end of the balloon is less than the diameter of the distal end of the balloon.

12. The catheter of claim 1, wherein the second balloon in the inflated state extends partially beyond the distal end of the shaft along the longitudinal axis of the shaft.

13. The catheter according to claim 1, wherein the first balloon in the inflated state has a diameter of 3 to 12 mm, preferably 5-10 mm, particularly preferably 7-8 mm.
